(19) United States
(12) Patent Application Publication
(10) Pub. No.: US 2019/0060407 A1
(43) Pub. Date: Feb. 28, 2019

(54) USE OF INTERLEUKIN 2 FOR TREATING SPONDYLOARTHRITIS

(71) Applicants: Assistance Publique - Hôpitaux De Paris, Paris (FR); ILTOO PHARMA, Paris (FR); SORBONNE UNIVERSITE, Paris (FR)

(72) Inventors: David Klatzmann, Paris (FR); Francis Berenbaum, Gif Sur Yvette (FR); Jérémie Sellam, Paris (FR); Bruno Fautrel, Paris (FR); Roberta Lorenzon, Paris (FR); Patrice Cacoub, Le Perreux (FR)

(73) Assignees: Assistance Publique - Hôpitaux De Paris, Paris (FR); ILTOO PHARMA, Paris (FR); SORBONNE UNIVERSITE, Paris (FR)

(21) Appl. No.: 15/758,800

(22) PCT Filed: Sep. 9, 2016
(86) PCT No.: PCT/EP2016/071370

§ 371 Date: Mar. 9, 2018

(30) Foreign Application Priority Data
Sep. 10, 2015 (EP) 15306383.9

(51) Int. Cl. A61K 38/20; A61P 19/02

(52) U.S. Cl. CPC A61K 38/2013; A61P 19/02

(57) ABSTRACT

The invention relates to the use of interleukin-2 in treating spondyloarthritis in a human subject, wherein IL-2 is to be administered at a dose of about I to about 2 MIU/day, wherein the treatment comprises at least a first course wherein interleukin-2 is administered once per day during at least 3 consecutive days, followed by a maintenance treatment after 1 to 4 weeks.

The present invention relates to administering interleukin 2 (IL-2) for use in treating spondyloarthritis. More specifically, the present invention relates to alleviating articular and extra-articular symptoms in patients with spondyloarthritis.

### BACKGROUND OF THE INVENTION

Spondyloarthritis (SpA) is a chronic inflammatory disease with either predominantly axial symptoms of the spine and sacroiliac joints (axial SpA, including ankylosing spondylitis) or predominantly arthritis (peripheral SpA) or both.

SpA primarily affects the spine, although other joints can become involved. It causes inflammation of the spinal joints (vertebrae) that can lead to severe, chronic pain and discomfort. In the most advanced cases (but not in all cases), this inflammation can lead to new bone formation on the spine, causing the spine to fuse in a fixed, immobile position, sometimes creating a forward-stooped posture. This forward curvature of the spine is called kyphosis.

SpA can also cause inflammation, pain and stiffness in other areas of the body such as the shoulders, hips, ribs, heels and small joints of the hands and feet.

Extra-articular manifestations vary widely in terms of both frequency and severity. The most common extra-articular manifestations are represented by uveitis, bowel disease, heart, lung, skin, bone, kidney involvement and fatigue.

The hallmark feature of SpA is the involvement of the sacroiliac (SI) joints during the progression of the disease, which are the joints at the base of the spine, where the spine joins the pelvis.

Unlike other forms of arthritis and rheumatic diseases, general onset of SpA commonly occurs in younger people, between the ages of 17-45. However, it can affect children and those who are much older. SpA is more common in men, but occurs in women as well.

The severity of SpA varies greatly from person to person, and not everyone will experience the most serious complications or have spinal fusion. Some will experience only intermittent back pain and discomfort, but others will experience severe pain and stiffness over multiple areas of the body for long periods of time. AS can be very debilitating, and in some cases, lead to disability.

Almost all cases of SpA are characterized by acute, painful episodes (also known as “flares”) followed by temporary periods of remission where symptoms subside.

Currently, there is no known cure for axial SpA. A standard managing treatment is nonsteroidal anti-inflammatory drugs (NSAIDs). Anti-TNFα in monotherapy or in combination with methotrexate, optionally with nonsteroidal anti-inflammatory drugs (NSAIDs) is used as second-line in case of intolerance or inefficacy of NSAIDs.

However there is still a need for more effective and safer drug in managing SpA.

### SUMMARY OF THE INVENTION

It is herein provided a method for treating SpA in a subject by administration of IL-2 at about 1 to about 2 MIU/day.

More specifically the invention provides IL-2 for use in treating spondyloarthritis in a subject, wherein IL-2 is to be administered at a dose of about 1 to about 2 MIU/day, wherein the treatment comprises at least a first course wherein interleukin-2 is administered once per day during at least 3 consecutive days, preferably during 3 to 7, still preferably during 4 to 5 consecutive days, preferably followed by a maintenance dose after 1 to 4 weeks.

This dosage and regimen effectively activate Tregs without substantially activating Teffs. The consequence is a dramatic increase in the Treg/Teff balance in the subject, without impact on its immunocompetency.

IL-2 is advantageously used in treating ankylosing spondylitis.

According to the invention, IL-2 is useful for alleviating at least one articular symptom associated with spondyloarthritis, such as arthralgia or morning stiffness, and/or at least one extra-articular symptom associated with spondyloarthritis, such as uveitis.

### DETAILED DESCRIPTION OF THE INVENTION

Definitions

The “subject” or “patient” to be treated may be any mammal, preferably a human being. The human subject may be a child, an adult or an elder. In other embodiments, the subject is a non-human mammal, such as cats, dogs, horses. The disease is often referred to “spondylosis deformans” or “cervical spondylosis deformans” in those non-human mammals.

The term “treating” or “treatment” means any improvement in the disease. It includes alleviating at least one symptom, or reducing the severity or the development of the disease.

In particular it includes reducing the risk, occurrence or severity of acute episodes (flares).

The term “treating” or “treatment” encompasses reducing the progression of the disease. In particular the invention encompasses preventing or slowing down the progression of SpA.

The term “treating” or “treatment” further encompasses prophylactic treatment, by reducing the risk or delaying the onset of the disease, especially in a subject who is asymptomatic but has been diagnosed as being “at risk”. The risk factors that predispose a person to SpA include:
Testing positive for the HLA-B27 marker
A family history of SpA

A personal or a family history of psoriasis, inflammatory bowel disease or uveitis
A personal history of reactive arthritis

“Regulatory T cells” or “Tregs” are T lymphocytes having immunosuppressive activity. Natural Tregs are characterized as CD4+CD25+Foxp3+ cells. Tregs play a major role in the control of inflammatory diseases, although their mode of action in such disease is not well understood. In fact, in most inflammatory diseases, Treg depletion exacerbates disease while Treg addition decreases it. Most Tregs are CD4+ cells, although there also exists a rare population of CD8+ Foxp3+ T lymphocytes with a suppressive activity.

Within the context of this application, “effector T cells” (or “Teff”) designates conventional T lymphocytes other than Tregs (sometimes also referred to as Tconv in the literature), which express one or more T cell receptor (TCR) and perform effector functions (e.g., cytotoxic activity, cytokine secretion, anti-self recognition, etc). Major populations of human Teff according to this invention include CD4+ T helper lymphocytes (e.g., Th0, Th1, Th17) and CD4+ or CD8+ cytotoxic T lymphocytes, and they can be specific for self or non-self antigens.

Spondyloarthritis (SpA)

The present invention relates to administering interleukin 2 (IL-2) for use in treating spondyloarthritis. More specifically, the present invention relates to alleviating articular and extra-articular symptoms in patients with spondyloarthritis.

Sites of involvement include the spine, peripheral joints, and entheses (capsules, ligaments, and tendons). The present invention more particularly aims at preventing or alleviating inflammatory enthesiopathy progressing to ossification and ankylosis.

Extra-articular symptoms include anterior uveitis, psoriasis or inflammatory bowel disease (IBD) and cardiovascular manifestations.

In a preferred aspect, it is provided a method for treating articular symptoms of spondyloarthritis in a patient in need thereof. In a particular embodiment the present invention aims at preventing or alleviating articular symptoms in patients with spondyloarthritis who do not show uveitis, or in patients with spondyloarthritis who do not show any extra-articular symptom.

Interleukin 2 (IL-2)

Within the context of this invention, the term “IL-2” designates any source of IL-2, including mammalian sources such as e.g., human, mouse, rat, primate, and pig, and may be native or obtained by recombinant or synthetic techniques, including recombinant IL-2 polypeptides produced by microbial hosts. IL-2 may be or comprise the native polypeptide sequence, or can be an active variant of the native IL-2 polypeptide. Preferably the IL-2 polypeptide or active variant is derived from a human source, and includes recombinant human IL-2, particularly recombinant human IL-2 produced by microbial hosts.

Active variants of IL-2 have been disclosed in the literature. Variants of the native IL-2 can be fragments, analogues, and derivatives thereof. By “fragment” is intended a polypeptide comprising only a part of the intact polypeptide sequence. An “analogue” designates a polypeptide comprising the native polypeptide sequence with one or more amino acid substitutions, insertions, or deletions. Muteins and pseudopeptides are specific examples of analogues. “Derivatives” include any modified native IL-2 polypeptide or fragment or analogue thereof, such as glycosylated, phosphorylated, fused to another polypeptide or molecule, polymerized, etc., or through chemical or enzymatic modification or addition to improve the properties of IL-2 (e.g., stability, specificity, etc.). Active variants of a reference IL-2 polypeptide generally have at least 75%, preferably at least 85%, more preferably at least 90% amino acid sequence identity to the amino acid sequence of the reference IL-2 polypeptide.

Methods for determining whether a variant IL-2 polypeptide is active are available in the art and are specifically described in the present invention. An active variant is, most preferably, a variant that activates Tregs.

Examples of IL-2 variants are disclosed, for instance, in EP109748, EP136489, U.S. Pat. No. 4,752,585; EP200280, or EP118617.

Preferably it is used a recombinant IL-2, i.e., an IL-2 that has been prepared by recombinant DNA techniques. The host organism used to express a recombinant DNA encoding IL-2 may be prokaryotic (a bacterium such as E. coli) or eukaryotic (e.g., a yeast, fungus, plant or mammalian cell). Processes for producing IL-2 have been described e.g., in U.S. Pat. No. 4,656,132; U.S. Pat. No. 4,748,234; U.S. Pat. No. 4,530,787; or U.S. Pat. No. 4,748,234, incorporated therein by reference.

In a preferred embodiment, the invention uses an IL-2 of human origin, or an active variant thereof, more preferably produced recombinantly. A nucleotide and an amino acid sequence of human IL-2 are disclosed, for instance, in Genbank access number 3558 or P60568, respectively. The invention more preferably uses a human IL-2.

IL-2 for use in the present invention is preferably in essentially pure form, e.g., at a purity of 95% or more, further preferably 96, 97, 98 or 99% pure.

For use in the present invention, IL-2 is typically not combined or co-administered with a Teff suppressive agent. However, although not preferred or required, drug combinations may be contemplated.

IL-2 may be used in monomeric or multimeric form.

IL-2 is commercially available, including for pharmaceutical uses, and it is authorized for use in human patients. Suitable commercial forms include, e.g.,
Proleukin® (aldesleukin) is a recombinant unglycosylated des-alanyl-1, serine-125 human interleukin-2, produced in E. coli.
Roncoleukin® is a recombinant human IL-2 produced in yeast.

In a preferred embodiment, IL-2 as used in the present invention is des-alanyl-1, serine-125 human interleukin-2, preferably produced recombinantly. In a particular embodiment it is unglycosylated, preferably it is produced in E. coli.

Interleukin-2 may be used alone or in combination with any other therapeutically active agent.

Dosage and Regimen

According to the invention, IL-2 is administered at a dosage ranging from about 1 MIU/day to about 2 MIU/day. This dosage is particularly suitable for human subjects.

This dosage effectively activates Tregs without substantially activating Teffs. The consequence is a dramatic increase in the Treg/Teff balance in the subject. At this dosage IL-2 substantially avoids side effects, while very substantially inducing Tregs.

In a preferred embodiment, particularly advantageous for subcutaneous administration, IL-2 is administered at a dose of 1, 1.5 or 2 MIU/day.

According to the invention, the treatment typically comprises at least a first course wherein interleukin-2 is administered once per day during at least 3 consecutive days, preferably during 3 to 7, still preferably during 4 to 5 consecutive days, preferably followed by a maintenance dose after 1 to 4 weeks.

The maintenance dose is typically administered during at least one month, preferably at least about 3 months, still preferably at least about 6 months. In a preferred embodiment, the maintenance dose is administered between about 3 months and about 12 months, preferably between about 6 months and about 12 months.

In a preferred embodiment, the maintenance treatment consists of an administration of interleukin-2 once or twice a week, every one or two weeks.

In a preferred embodiment, the maintenance treatment consists of an administration of interleukin-2 once or twice a week, every one or two weeks, during a period of at least one month, preferably from about 3 months to about 12 months.

Preferably the maintenance dosage is substantially the same as the first course dosage, or it can be a lower dosage.

In a preferred embodiment, the treatment comprises at least a first course wherein interleukin-2 is administered at a dosage of about 1 to about 2 MIU/day, preferably 1-1.5 MIU/day once per day during 3 to 7 days, preferably 5 days, followed by a maintenance dose after two weeks, of about 1 to about 2 MIU/day, preferably 1-1.5 MIU/day every 2 weeks, during at least three months, preferably at least six months.

In a particular embodiment, the subject is administered with IL-2 as the single active ingredient effective in treating spondyloarthritis.

In another particular embodiment, the subject it administered with IL-2, as well as with other active ingredients, either simultaneously or sequentially. For instance, the subject may be administered with IL-2 in combination with an anti-Tumor necrosis Factor (TNF) compound, especially anti-TNFα antibody, or methotrexate, and/or with nonsteroidal anti-inflammatory drugs (NSAIDs). However, in preferred embodiments, the dosage of such additional active ingredients can be reduced dramatically, reducing the risk and severity of side effects.

Administration Forms and Routes

Il-2 may be administered using any convenient route, including parenteral, e.g. intradermal, subcutaneous, or intranasal route. The subcutaneous route is preferred. Oral, sublingual or buccal administrations are also encompassed.

IL-2 is typically administered in association (e.g., in solution, suspension, or admixture) with a pharmaceutically acceptable vehicle, carrier or excipient. Suitable excipients include any isotonic solution, saline solution, buffered solution, slow release formulation, etc. Liquid, lyophilized, or spray-dried compositions comprising IL-2 or variants thereof are known in the art and may be prepared as aqueous or nonaqueous solutions or suspensions. Preferably the pharmaceutical compositions comprise appropriate stabilizing agents, buffering agents, bulking agents, or combinations.

The Examples illustrate the invention without limiting its scope.

### EXAMPLES

Patient Selection

Inclusion criteria for study were as follows: 1) documented diagnosis of SpA according with ASAS criteria, 2) moderately active disease (30≤BASDAI≤60), 3) under standard treatment (≥2 months) at the time of inclusion (anti-TNFα in monotherapy or in combination with Methotrexate+/−NSAID). ASAS is Assessment of SpondyloArthritis International Society is intended for classification of both axial and peripheral SpA (Rudwaleit M, van der Heijde D, Landewé R et al. The assessment of SpondyloArthritis International Society classification criteria for peripheral spondyloarthritis and for spondyloarthritis in general. Ann. Rheum. Dis. 70(1), 25-31 (2011)). BASDAI (Bath Ankylosing Spondylitis Disease Activity Index) is described in GARRETT et al. A new approach to defining disease status in ankylosing spondylitis: the Bath Ankylosing Spondylitis Disease Activity Index. J Rheumatol 1994 21 (12) 2286-2291.

Exclusion criteria included co-infection with HBV or HIV, several organ damages (heart failure, renal insufficiency, or hepatic insufficiency, or lung failure), pregnancy and drug addiction.

Study Design

A multicentric, uncontrolled, open-label phase II study, comparing biological and clinical responses to the administration of low doses IL-2 (prepared from Proleukin®).

Each patient received 1 MUI/day of IL2 from Day-1 to Day-5 (the induction period), and then every 2 weeks from Day-15 to Day-180 (the maintenance period). Patients are then followed up for 2 months (Day-240).

Primary efficacy endpoint is the Treg response at Day-8. Secondary endpoints are:
Treg response during the maintenance period,
changes in markers of inflammation
clinical response, evaluated by means of global generic scales [Clinical Global Impression severity scale (CGI-sev) and Clinical Global Impression efficacy index (CGI-eff)] as well as specific clinical and biological evaluations for each disease (CRP C-reactive protein, CRPus C-reactive protein ultrasensitive, BASDAI for SpA)
frequency of relapses,
assessment of quality of life (scale EuroQL-5).

### RESULTS

Patient 1-02-02-C-L: (axial SpA, HLA-B27+) male, 38 years old. Regular treatment: anti-TNFα and NSAID.

Under IL-2 therapy, the patient has dramatically decreased his BASDAI score (from 43.5/100 at baseline to 14/100 after 6 months of IL-2 therapy, notably due to decrease of arthralgia, asthenia and morning stiffness. This clinical benefit was maintained 2.5 months after treatment discontinuation (BASDAI score=14/100).

Uveitis episodes have decreased in frequency and intensity. Patient describes also an increase of physical performance in sport activities. Due to this clinical improvement, the patient has stopped intake of NSAID.
Patient 1-02-05-G-M: (peripheral SpA, HLA-B27+) male, 65 years old. Regular treatment: with methotrexate and NSAID.

Under IL-2 therapy the patient has dramatically decreased his BASDAI score (from 46/100 at baseline to 4/100 after 6 months of IL-2 therapy), notably due to decrease of arthralgia, asthenia and morning stiffness. This clinical benefit was maintained 2.5 months after treatment discontinuation (BASDAI score=10/100).

In the same time, ESR (erythrocyte sedimentation rate) value has decreased and has returned to normal values.

Patient describes also an increase of physical performance in sport activities “I feel like I am twenty years old”.

After 6 months with IL-2 therapy, the patient has begun to participate to marathons.

For this clinical improvement the patient has stopped to take NSAID.
Patient 2-02-03-S-S: (axial SpA, HLA-B27+) male, 25 years old. Regular treatment: anti-TNFα and NSAID.

Under IL-2 therapy the patient has dramatically decreased BASDAI score (from 35.9/100 at baseline to 12.1 after 6 months of IL-2 therapy), notably due to decrease of arthralgia, asthenia and morning stiffness. This clinical benefit was maintained 2.5 months after treatment discontinuation (BASDAI score=8.3/100).

In the same time, his CRP-value has decreased under IL2 therapy.
Patient 2-02-04-ED: (axial and peripheral SpA, Crohn) male, 47 years old. Regular treatment: anti-TNFα.

The patient describes a clinical benefit under IL-2 therapy during 10 days after each TL-2 administration.
Patient 2-02-06-L-A: (peripheral SpA, HLA B27−) male, 43 years old.

This patient has an initial clinical benefit manifested by resuming sport activity.
Patient 1-07-01-C-D: (axial and peripheral SpA, Takayasu disease and Ulcerative Colitis) female, 50 years old. Regular treatment: corticosteroids (7 mg/d), methotrexate (15 mg/w) and paracetamol (acetaminophen).

Under IL-2 therapy the patient has decreased BASDAI score (from 45.5/100 at baseline to 31/100 after 3 months of IL-2 therapy), notably due to decrease of arthralgia, asthenia and morning stiffness. The patient has stopped the intake of paracetamol.

This clinical benefit allowed the decrease the weekly dose of methotrexate. The patient reports long walk without muscular pain.

This clinical benefit was maintained 2.5 months after treatment discontinuation (BASDAI score=18/100) and, this allowed the decrease of the daily dose of corticosteroids.
Patient 1-05-02-V-D: (axial and peripheral SpA, HLA B27+, Behçet disease) male, 50 years old. Regular treatment: corticosteroids, colchicine, and analgesic drug.

Under IL-2 therapy, the patient has dramatically decreased his BASDAI score (from 31/100 at baseline to 13/100 after 3 months of IL-2 therapy), notably due to decrease of arthralgia, asthenia and morning stiffness. The patient has stopped intake of analgesic drug. This clinical benefit was maintained 2.5 months after treatment discontinuation (BASDAI score=19/100).
2-02-10-R-F: (mixt SpA, HLA B27+) male, 42 years old. Regular treatment : NSAID.

Under IL-2 therapy the patient has decreased BASDAI score (from 43/100 to 30.5/100 after 3 months) notably due to decrease of asthenia and morning stiffness.

1. Interleukin-2 for use in treating spondyloarthritis in a subject, wherein IL-2 is to be administered at a dose of about 1 to about 2 MIU/day, wherein the treatment comprises at least a first course wherein interleukin-2 is administered once per day during at least 3 consecutive days, followed by a maintenance treatment after 1 to 4 weeks.

2. Interleukin-2 for use according to claim 1, wherein spondyloarthritis is ankylosing spondylitis.

3. Interleukin-2 for use according to claim 1 or 2, for alleviating at least one articular symptom associated with spondyloarthritis.

4. Interleukin-2 for use according to claim 3, wherein the articular symptom is arthralgia or morning stiffness.

5. Interleukin-2 for use according to claim 1 or 2, for alleviating at least one extra-articular symptom associated with spondyloarthritis.

6. Interleukin-2 for use according to claim 5, wherein the extra-articular symptom is uveitis.

7. Interleukin-2 for use according to any of claims 1 to 6, wherein it is to be administered at a dose of about 1-1.5 MIU/day.

8. Interleukin-2 for use according to any of claims 1 to 3, wherein IL-2 is administered repeatedly.

9. Interleukin-2 for use according to any of claims 1 to 8, wherein the treatment comprises at least a first course wherein interleukin-2 is administered once per day during 3 to 7 days, during 4 to 5 consecutive days, followed by the maintenance dose after 1 to 4 weeks.

10. Interleukin-2 for use according to any of claims 1 to 9, wherein the maintenance treatment consists of an administration of interleukin-2 once or twice a week, every one or two weeks, during a period of at least one month, preferably from about 3 months to about 12 months.

11. Interleukin-2 for use according to any of claims 1 to 10, wherein interleukin-2 is administered by subcutaneous route.

12. Interleukin-2 for use according to any of claims 1 to 11, wherein the treatment is preventive, the subject being susceptible to develop spondyloarthritis.

13. Interleukin-2 for use according to any of claims 1 to 11, wherein the treatment reduces the number and/or severity of inflammatory episodes.

14. Interleukin-2 for use according to any of claims 1 to 13, wherein the subject is human.

15. Interleukin-2 for use according to any of claims 1 to 14, wherein the maintenance treatment consists of an administration of interleukin-2 once or twice a week, every one or two weeks, during a period of at least one month, preferably from about 3 months to about 12 months.
